(19) United States
(12) Patent Application Publication
(10) Pub. No.: US 2026/0151157 A1
(43) Pub. Date: Jun. 4, 2026

(54) CATHETER PLACEMENT DEVICES

(71) Applicant: Board of Regents of the University of Nebraska, Lincoln, NE (US)

(72) Inventors: Mark CARLSON, Omaha, NE (US); Keely BUESING, Omaha, NE (US); Benjamin TERRY, Lincoln, NE (US); Benjamin WANKUM, McCook Lake, SD (US); Riley REYNOLDS, Lincoln, NE (US); Sean CRIMMINS, Lincoln, NE (US)

(21) Appl. No.: 18/281,911

(22) PCT Filed: Mar. 16, 2022
(86) PCT No.: PCT/US2022/020534

§ 371 Date: Sep. 13, 2023

Related U.S. Application Data
(60) Provisional application No. 63/162,178, filed on Mar. 17, 2021.

(51) Int. Cl. A61B 17/34

(52) U.S. Cl. CPC A61B 17/3415; A61B 17/3423; A61B 17/3474; A61B 17/3498

(57) ABSTRACT

Apparatuses and methods can be used to install a catheter or trocar in a patient to gain access to an internal region of the patient, such as the peritoneal cavity. For example, the devices described herein provide a non-electric, mechanical means to access the peritoneal space. The devices are designed for safe and reliable placement of a peritoneal access catheter even in austere conditions, such as on the battlefield or in another type of emergency setting. The devices are designed with safety being paramount, and are therefore capable of providing low enough pressure to allow access without causing pre-peritoneal insufflation, yet high enough to separate abdominal viscera from the parietal peritoneum. The devices are designed to be straightforward to use, and only require minimal training for use.

### CROSS-REFERENCE TO RELATED APPLICATIONS

This application claims priority under 35 U.S.C. § 119 to U.S. Provisional Application Ser. No. 63/162,178, filed on Mar. 17, 2021, which is incorporated by reference in its entirety herein.

### STATEMENT AS TO FEDERALLY SPONSORED RESEARCH

This invention was made with government support under STRATCOM award number FA460018D9001; FU911 awarded by the Department of Defense—Offutt Air Force Base. The government has certain rights in the invention.

### TECHNICAL FIELD

This disclosure relates to apparatuses and methods for installing a catheter or trocar in a patient to gain access to an internal region of the patient, such as the peritoneal cavity, for laparoscopic surgery and/or other medical purposes.

### BACKGROUND

Trocars are placed through the abdominal wall during laparoscopic surgery. Thereafter, the trocar functions as a portal for the subsequent placement of other instruments, such as graspers, scissors, staplers, endoscopes, etc. In some cases, the trocar can be used as an insufflation gas conduit to insufflate the peritoneal cavity or other internal regions of the body.

Peritoneal cavity access is essential in several medical procedures and surgeries. For example, accessing the peritoneal cavity can be justified for a variety of medical procedures including oxygen microbubble infusion, nephrectomy, hysterectomy, cholecystectomy, colectomy, bariatric surgery, adrenalectomy, anti-reflux, abdominal perineal resection, hernia repair, gastrectomy, and appendectomy. Intraperitoneal cavity infusion of oxygen microbubbles (OMBs) is a means of oxygenating the body when the lungs are not capable of doing so due to a variety of health issues (e.g., acute respiratory distress syndrome, smoke inhalation, or gunshot wound). To infuse the microbubbles, the peritoneal cavity must first be accessed, and a catheter must safely be placed within the cavity.

Most complications and adverse events during laparoscopic surgery occur during initial entry into the peritoneal cavity. Among them, pre-peritoneal insufflation occurs when the insufflation needle is incorrectly placed, and the abdominal wall is insufflated. The complications related to accessing the peritoneal cavity are amplified under austere conditions, such as on the battlefield or in an ambulance.

With over 13 million laparoscopic procedures performed globally and a primary access complication rate of around one in 1000, over 13,000 complications occur per year. Healthcare practitioners must be ever mindful during initial entry as most life-threatening accidents to the great vessels or the intestines occur during initial access.

Moreover, there are current devices that are designed for peritoneal cavity access procedures under operating room conditions. However, for conditions outside the controlled environment of the hospital operating room (e.g., the battlefield), no devices have been successfully developed.

### SUMMARY

This disclosure describes apparatuses and methods for installing a catheter or trocar in a patient to gain access to an internal region of the patient, such as the peritoneal cavity. For example, the devices described herein provide a non-electric, mechanical means to allow placement of surgical instruments into the peritoneal space. The devices described herein are designed for safe and reliable placement of a peritoneal access catheter in austere conditions, such as on the battlefield or other emergency situations away from a clinical setting. The devices are designed with safety being paramount, and are therefore capable of providing low enough gas pressure to allow access without causing pre-peritoneal insufflation, yet high enough to separate abdominal viscera from the parietal peritoneum. These features are described further below.

In one aspect, this disclosure is directed to a medical device that includes a catheter defining a lumen extending along a longitudinal axis of the catheter; a compressed gas source; a valve member having: (i) a closed position in which the valve member blocks the compressed gas source from being in fluid communication with the lumen and (ii) an open position that allows fluid communication between the compressed gas source and the lumen; and a rotary actuator that, when driven, rotates the catheter about its longitudinal axis.

Such a medical device may optionally include one or more of the following features. The catheter may have a helical protrusion on an outer surface of the catheter. The rotary actuator may include a hand crank by which the catheter is manually rotatable about its longitudinal axis. The valve member may be manually movable between the closed and open positions. The compressed gas source may include a bellows and a spring. The bellows and the spring may be arranged such that the spring resists an expansion of the bellows to draw air into the bellows. The bellows may be manually expandable. In some embodiments, when: (i) the bellows is expanded, (ii) the valve member is in the open position, and (iii) a distal end of the lumen is occluded, the spring compresses the bellows to create a gauge pressure of 16 mmHg to 20 mmHg within the lumen.

In another aspect, this disclosure is directed to a method of accessing an internal region of a mammalian body. The method includes: making a skin incision; placing a distal tip portion of a trocar through the skin incision and advancing the trocar a first distance into tissue; after advancing the trocar the first distance, increasing a gas pressure in a lumen of the trocar; after increasing the gas pressure in the lumen, advancing the trocar farther into the tissue until a decrease of the gas pressure in the lumen is noticeable; and advancing a surgical instrument through the lumen and into the internal region via the lumen of the trocar. The decrease of the gas pressure in the lumen indicates that the lumen has become in fluid communication with the internal region.

Such a method may optionally include one or more of the following features. The advancing the trocar into the tissue may be performed by rotating the trocar about its longitudinal axis such that helical protrusions on an outer surface of the trocar drill into the tissue. The increasing the gas pressure in the lumen may involve opening a valve member to put a bellows and the lumen of the trocar in fluid communication with each other. The method may also include, prior to opening the valve member, expanding the bellows to: (i) cause gas to flow into the bellows and (ii) add loading to a spring. The method may also include, after expanding the bellows, closing the valve member prior to advancing the trocar farther into the tissue. The decrease of the gas pressure in the lumen may cause the trocar to no longer be rotatable. The gas pressure may be increased to a gauge pressure of 16 mmHg to 20 mmHg within the lumen of the trocar.

In another aspect, this disclosure is directed to a trocar installation device that includes a trocar defining a lumen extending along a longitudinal axis of the trocar, and a compressed gas source that is manually actuatable to selectively pressurize the lumen.

Such a trocar installation device may optionally include one or more of the following features. The trocar may include a helical protrusion on an outer surface of the trocar. The trocar installation device may also include a manual hand crank mechanism that is operable to rotate the trocar about its longitudinal axis. The compressed gas source may include a bellows and a spring. The trocar installation device may also include a latch mechanism that is operable to releasably detain the bellows in an expanded configuration and the spring in a loaded configuration. The compressed gas source may comprise a pressurized bottle containing the gas.

Some embodiments described herein may provide one or more of the following advantageous benefits. Current conventional technologies for peritoneal access involve a surgery room (e.g., adequate lighting, vital monitoring, laparoscopic scopes, insufflator, etc.), a physician, and necessary anesthetics/medication. The devices and methods described herein remove the need for these and only require minimal training to perform. No electricity is required to use the devices and methods described herein. Accordingly, the devices and methods can be used in virtually any setting including, for example, on a battlefield and other emergency scenarios. In some embodiments, the devices described herein include an indication mechanism that notifies the user when the distal tip portion of the cannula has broken through a body wall (e.g., the abdominal wall) and is positioned just within a body cavity (e.g., the peritoneal cavity). The devices described herein are thereby designed to prevent catheter plunging, and the associated risk of organ damage, that can occur with some conventional catheters when the catheter breaks through a body wall to access a body cavity.

It is appreciated that methods in accordance with the present disclosure may include any combination of the aspects and features described herein. That is, methods in accordance with the present disclosure are not limited to the combinations of aspects and features specifically described herein, but also include any combination of the aspects and features provided.

The details of one or more implementations of the subject matter of this disclosure are set forth in the accompanying drawings and the description below. Other features, objects, and advantages of the subject matter will be apparent from the description and drawings, and from the claims.

### BRIEF DESCRIPTION OF DRAWINGS

FIG. 1 illustrates a cannula or trocar that has been advanced through an abdominal wall (shown in cross-section) so that the distal tip of the cannula or trocar is in a peritoneal cavity.

FIG. 2 is a perspective view of an example catheter placement device in accordance with some embodiments described herein.

FIG. 3 is a magnified view of a distal tip portion of the catheter of the example catheter placement device of FIG. 2.

FIG. 4 is a longitudinal cross-section view of the example catheter placement device of FIG. 2.

FIG. 5 is an exploded side view of the example catheter placement device of FIG. 2.

FIG. 6 is an exploded perspective view of the example catheter placement device of FIG. 2.

FIG. 7 is a flowchart of an example method for accessing an internal region of a mammalian body using the catheter placement devices described herein.

### DETAILED DESCRIPTION

Apparatuses and methods as described herein can be used for installing a catheter, cannula, or trocar in a patient to gain access to an internal region of the patient, such as the peritoneal cavity. In some embodiments described herein, the devices provide a non-electric, mechanical means to access the peritoneal cavity. Moreover, the devices are designed for safe and reliable placement of an access catheter in austere conditions, such as on the battlefield or other types of non-clinical emergency settings. The devices are designed with safety being paramount, and are therefore capable of providing low enough pressure to indicate successful access without causing pre-peritoneal insufflation, yet high enough to separate abdominal viscera from the parietal peritoneum, as described further below.

Multiple embodiments of catheter placement devices are described herein. Certain features that are described in the context of a particular embodiment may also be implemented in combination with the features of another embodiment. Conversely, various features that are described in the context of a single embodiment may also be implemented in multiple implementations separately or in any suitable sub-combination. Although features may be described below as acting in certain combinations and even initially claimed as such, one or more features from a claimed combination may in some examples be excised from the combination, and the claimed combination may be directed to a sub-combination or variation of a sub-combination.

Certain devices are referred to herein using various terms such as “catheter,” “cannula,” or “trocar” and are considered to be interchangeable for the purposes of this disclosure. Each such “catheter,” “cannula,” or “trocar” device comprises, for example, an elongate tubular device that defines at least one longitudinally-extending lumen. The lumen can be used to provide access to the internal body space by medical instruments, for insufflation of the internal body space, and various other purposes.

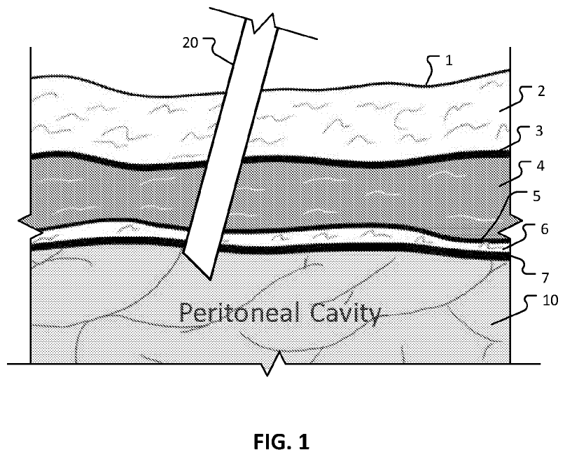

Referring to FIG. 1, an example catheter 20 can be used to provide access to a peritoneal cavity 10 (e.g., for laparoscopic surgery). While this example illustrates an abdominal wall and peritoneal cavity 10, it should be understood that the catheter placement devices described herein are not limited to the illustrated use context. In other words, the catheter placement devices described herein can also be used in other body areas such as, but not limited to, the thoracic space, the GI tract, within the lungs, the pericardial space, intrauterine space, and so on, without limitation.

In this example, the catheter 20 has been inserted into the peritoneal cavity 10 through an incision in the skin 1 of the abdomen. Just beneath the skin 1, is a layer of subcutaneous fat 2. Below the subcutaneous fat 2 in this example is an anterior rectus sheath 3. Just below the anterior rectus sheath 3 is a layer of one or more abdominal muscles 4. Below the layer of muscles 4 is a posterior rectus sheath 5. Below the posterior rectus sheath 5 is another layer of fat, i.e., the peritoneal fat 6. Finally, between the peritoneal fat 6 and the peritoneal cavity 10 is a parietal peritoneum 7.

In contrast to the catheter placement devices described herein, conventional catheters can present some patient risks and use challenges when used to access body cavities such as the peritoneal cavity 10. For example, in some cases one or more of the layers of tissue below the skin 1 can have a high modulus of elasticity. Accordingly, a sudden release of resistance to the advancement of the catheter 20 may tend to occur when breaking through one or more layers of tissue (e.g., the parietal peritoneum 7). In such a case, the catheter 20 may tend to plunge into the body cavity in an inadvertent manner. Organ damage, perforation, bleeding, and other complications can sometimes result. These risks are mitigated by the catheter placement devices described herein, as described further below.

In some cases, the catheter 20 is used to deliver concomitant insufflation during the advancement of the catheter 20. In such a case, there can be an undesirable potential for the gas pressure of the insufflation to separate the tissue, or the layers of tissue, of the body wall. For example, as the catheter 20 is being advanced, and while being pressurized for the sake of providing concomitant insufflation, in some cases the layer of muscles 4 may become separated and then receive an amount of the insufflation gas within the layer of muscles 4. This result is undesirable. These risks are mitigated by the catheter placement devices described herein, as described further below.

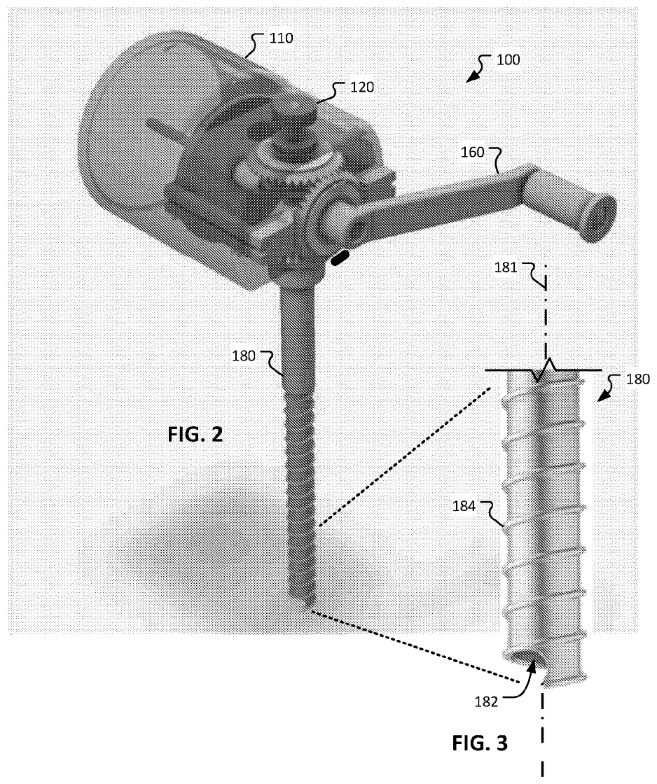

FIG. 2 illustrates an example catheter placement device 100. Broadly speaking, the catheter placement device 100 includes a housing 110 (containing other components as described below), a valve actuator knob 120, a rotary actuator 160, and a catheter 180. The catheter placement device 100 can be used to advance the catheter 180 through a body wall so that the distal tip of the catheter 180 is positioned in a body cavity, thereby providing access to the body cavity for medical instruments through a lumen of the catheter 180.

The depicted catheter placement device 100 is designed to be fully self-contained. That is, there is no need to connect the catheter placement device 100 to a source of electricity, gas, vacuum, liquid, or any other typical medical device connection.

The rotary actuator 160 (depicted as a hand crank in this example) can be rotated (e.g., manually rotated) to cause the catheter 180 to be rotated. That is the case because the rotary actuator 160 is coupled to the catheter 180 by a gear train, as described further below. The rotation of the catheter 180 allows the catheter 180 to be advanced through the tissue of a body wall.

FIG. 3 provides an enlarged view of a distal tip portion of the catheter 180. The catheter 180 defines a longitudinal axis 181 and a lumen 182 that extends along the longitudinal axis 181. Additionally, the catheter 180 includes a helical protrusion 184 radially protruding from an outer surface of the catheter 180. As the rotary actuator 160 is driven (e.g., as the hand crank is manually rotated), the catheter 180 rotates about its longitudinal axis 181.

The helical protrusion 184 on the outer surface of the catheter 180 configures the catheter 180 to act like a drill. Accordingly, as the catheter 180 is rotated, it can be readily advanced through the tissue of a body wall. No excessive pushing of the catheter 180 is required because the helical protrusion 184 on the outer surface of the catheter 180 tends to advance the catheter 180 distally through tissue as the catheter 180 is rotated.

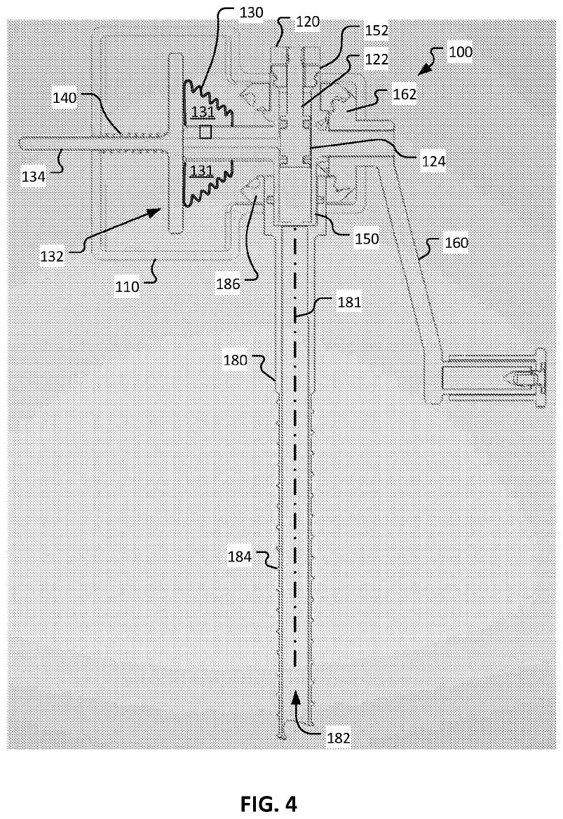

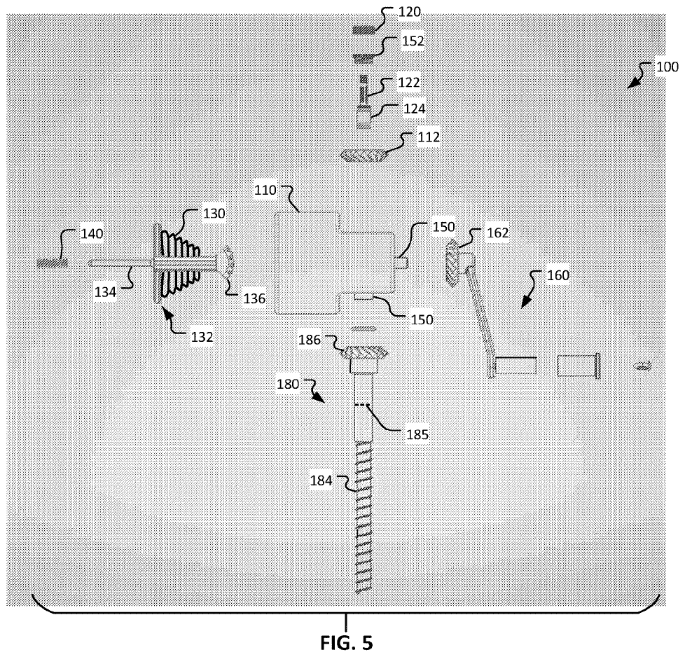

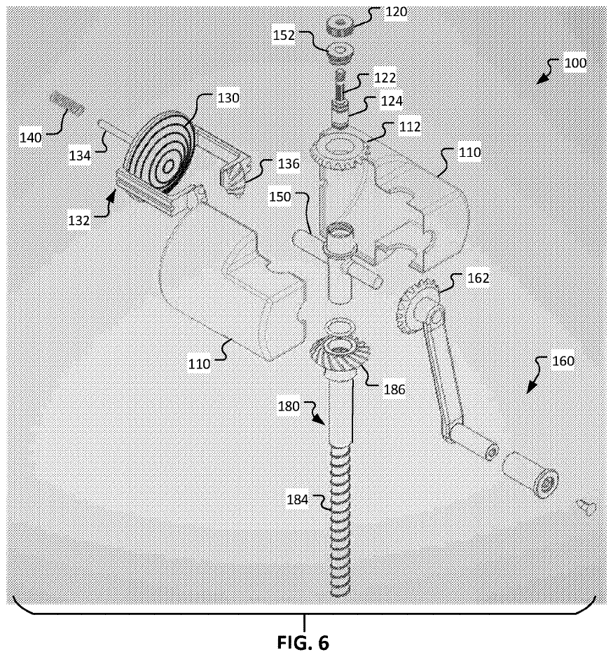

FIGS. 4-6 provide additional views of the catheter placement device 100. FIG. 4 provides a longitudinal cross-sectional view, and FIGS. 5 and 6 provide exploded views. In these views, additional components of the catheter placement device 100 are visible. For example, it can be seen that the catheter placement device 100 includes a bellows 130, a spring 140, and an axle member 150. In addition, it can be seen that the valve actuator knob 120 is connected to a valve stem 122, and the valve stem 122 is connected to a valve member 124. The valve member 124 can include one or more fluid seals (e.g., o-rings) as needed to function as a selectively operable fluid sealing device.

The axle member 150 is a multipurpose component of the catheter placement device 100. For example, the axle member 150 serves both as a mechanical axle and as a fluidic valve housing. Regarding the mechanical axle aspect of the axle member 150, the rotary actuator 160 is rotatably coupled to the axle member 150. In addition, the catheter 180 is rotatably coupled to the axle member 150. Further, the rotary actuator 160 and the catheter 180 are coupled together by a gear train comprising beveled gears (e.g., a drive bevel gear 162 attached to the rotary actuator 160 and a driven bevel gear 186 attached to the catheter 180). Accordingly, as the rotary actuator 160 is rotated, both the rotary actuator 160 and the catheter 180 rotate about the axle member 150 (which functions like two axles that are oriented at 90° apart from each other).

Regarding the valve housing aspect of the axle member 150, the bellows 130 and the catheter 180 are each mechanically coupled to the axle member 150 and arranged to be in fluid communication with each other via the axle member 150. Depending on the position of the valve member 124 within the axle member 150, the bellows 130 can be in fluid communication with the lumen 182 of the catheter 180, or can be blocked from being in fluid communication with each other (again, as determined by the position of the valve member 124 which is manually movable using the valve actuator knob 120).

The valve member 124 is movably coupled within the axle member 150. The valve member 124 can be manually moved relative to the axle member 150 by pulling the valve actuator knob 120 away from the axle member 150, and by pushing the valve actuator knob 120 toward the axle member 150. In that manner the valve member 124 is reconfigurable between: (i) a closed position in which the valve member 124 blocks the bellows 130 from being in fluid communication with the lumen 182 of the catheter 180 and (ii) an open position that allows fluid communication between the bellows 130 and the lumen 182 of the catheter 180. Pushing the valve actuator knob 120 toward the axle member 150 moves the valve member 124 to its closed position, and pulling the valve actuator knob 120 away from the axle member 150 moves the valve member 124 to its open position. Accordingly, a user of the catheter placement device 100 can selectively open and close the valve member 124 by manually manipulating the position of the valve actuator knob 120.

The bellows 130 is an expandable elastomeric member that defines an internal space 131 (FIG. 4). The bellows 130 can be expanded to increase the size of the internal space 131, and can be compressed to decrease the size of the internal space 131. When the bellows 130 is expanded, air is naturally drawn into the internal space 131. When the bellows 130 is compressed, air is naturally expelled from the internal space 131. For example, assuming the valve member 124 is in its open position, the action of expanding the bellows 130 causes air to be drawn into the lumen 182 of the catheter 180, which then flows through the axle member 150 and into the internal space 131 of the bellows 130. Conversely, assuming the valve member 124 is in its open position, the action of compressing the bellows 130 causes air to be expelled from the internal space 131, which then flows through the axle member 150 and into the lumen 182 of the catheter 180. The air can exit the distal end of the catheter 180 assuming that the lumen 182 is not blocked at the distal end of the catheter 180.

The bellows 130 is mounted to a support member 132 and connected to the axle member 150 (which is a fluidly sealed connection). The support member 132 is movable relative to the housing 110 and to the axle member 150. Moving the support member 132 away from the axle member 150 causes the bellows 130 to expand. Moving the support member 132 toward the axle member 150 causes the bellows 130 to be compressed.

The support member 132 includes a shaft 134. A free end portion of the shaft 134 extends outside of the housing 110 (e.g., as best seen in FIG. 4). The free end portion of the shaft 134 can be manually grasped and pulled to cause the bellows 130 to expand (which, in turn, draws air into the bellows 130 assuming that the valve member 124 is in its open position).

The catheter placement device 100 also includes a spring 140. The spring 140 is a coil compression spring. The spring 140 is mounted on the shaft 134, and abuts against an internal wall of the housing 110. Accordingly, as the free end portion of the shaft 134 is pulled to expand the bellows 130, the spring 140 becomes compressed (or loaded) to store potential energy. It can also be said that the spring 140 resists the action of expanding the bellows 130. The spring 140 naturally wants to push the bellows 130 to its compressed arrangement (which would expel the air from the internal space 131 of the bellows 130).

The combination of the bellows 130 and the spring 140 can be manipulated to function as a compressed gas source. For example, air can be drawn into the internal space 131 of the bellows 130 by pulling on the shaft 134 while the valve member 124 is in its open position. While the bellows 130 remains in its expanded configuration, the valve member 124 can be closed. After the valve member 124 is closed, then the shaft 134 can be released. When the shaft 134 is released, the spring 140 will compress the bellows 130 (which will compress the air in the internal space 131 to a pressure that is higher than ambient pressure). However, the valve member 124 (in its closed position) will not allow the air from the bellows 130 to flow into the lumen 182 of the catheter 180. Therefore, the air in the internal space 131 of the bellows 130 will become compressed air (having a positive gage pressure, i.e., a pressure that is higher than the ambient air pressure). The spring rate of the spring 140 and the volume of the internal space 131 of the bellows 130 will determine the gage pressure of the air in the internal space 131.

In summary, the following statements describe the main functional aspects of the catheter placement device 100. The catheter 180 can be rotated about its longitudinal axis 181 by manually turning the rotary actuator 160. The helical protrusions 184 on the catheter 180 will cause the catheter 180 to function as a drill while the catheter 180 is being rotated. The valve member 124 can be manually moved between an open position and a closed position by pulling or pushing the valve actuator knob 120. The bellows 130 can be expanded by manually pulling on the shaft 134. The bellows 130 will be compressed when the spring 140 releases its compressive force against the support member 132.

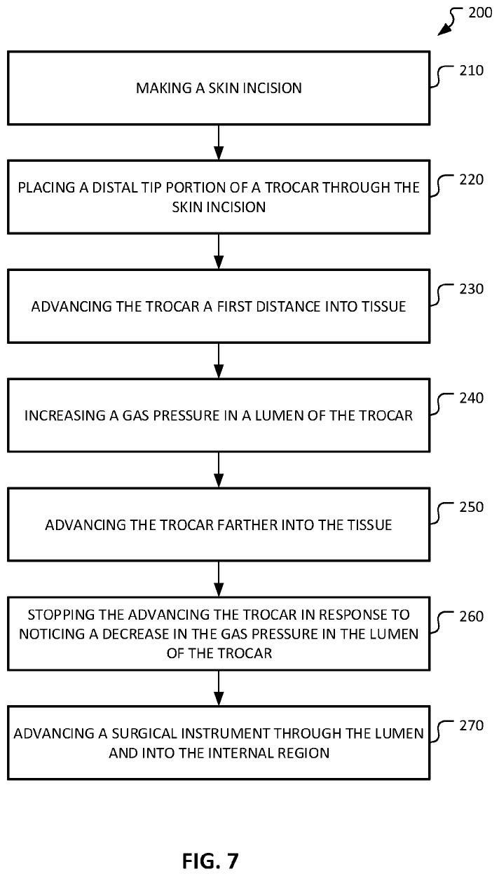

FIG. 7 is a flowchart that depicts a method 200 of accessing an internal region of a mammalian body. The method 200 can be performed using the catheter placement device 100 described above, and explanatory references will be made thereto. The method 200 can be performed by a “user” who may be a healthcare practitioner, or may be a layman with minimal training. The method 200 may be performed in virtually any setting, including austere settings (e.g., a battlefield and other non-clinical settings), and clinical settings. The method 200 may performed on a human patient and on other mammalian patients.

In step 210 of the method 200, an incision is made through a skin layer of the patient. For example, in the non-limiting example implementation of laparoscopic abdominal surgery on a human patient, an incision can be made in the skin of the patient near the navel.

In step 220, the user can insert a distal tip portion of a trocar through the skin incision. For example, the distal tip portion of the catheter 180 of the catheter placement device 100 described above can be placed through the skin incision. At this stage of using the catheter placement device 100, the compressed air source is loaded. That is: (i) the bellows 130 is in its expanded configuration, (ii) the valve member 124 is in its closed position, and (iii) the spring 140 is compressing the bellows 130. Accordingly, the air inside of the bellows 130 is residing at a positive gage pressure (compressed air).

In step 230, the trocar is advanced a first distance into the tissue of the body wall. The first distance is a distance that results in the creation of a fluid seal between the lumen 182 of the catheter 180 and the tissue. In some cases, the first distance is about 5 mm, or between 3 mm and 7 mm, or between 2 mm and 10 mm, without limitation.

In step 240, the gas pressure in the lumen of the trocar is increased. To achieve this in the context of using the catheter placement device 100 described above, for example, the valve member 124 can be manually moved to its open position. When the valve member 124 is moved to its open position, the compressed air in the bellows 130 will be released to flow into the lumen 182 of the catheter 180. Since the distal end of the catheter 180 is fluidly sealed by the tissue of the body wall, the compressed air will not flow out of the catheter 180, and therefore the pressure of the air in the lumen 182 will be increased. In addition, when the valve member 124 is moved to its open position, the spring 140 will release at least some of its stored energy, the bellows 130 will at least partially compress, and the support member 132 will move (translate) toward the axle member 150.

It is beneficial to control the gage pressure of the gas in the lumen of the trocar resulting from step 240 to be maintained within a narrow range of gage pressure. If the pressure of the gas in the lumen is too high, the gas may be undesirably injected into the body wall itself (e.g., between layers of the body wall, or within a layer of the body wall). Conversely, if the pressure of the gas in the lumen is too low, the user may not be able to notice when the distal tip of the trocar breaks through the body wall into the body cavity (as described further below).

In step 250, the user continues gradually advancing the trocar farther into the tissue of the body wall. This can be performed by manually rotating the rotary actuator 160 of the catheter placement device 100 described above, for example. The catheter 180 will continue to drill deeper into the body wall as a result.

At step 260, the user stops advancing the trocar. In particular, the user stops advancing the trocar in response to noticing that the pressure of the gas in the lumen of the trocar has experienced a significant decrease. The significant decrease of the pressure of the gas in the lumen of the trocar can be used to indicate that the distal tip of the trocar has broken through the body wall such that the lumen of the trocar has become positioned in fluid communication with the body cavity. The body cavity exerts less resistance to the flow of the gas from the lumen of the trocar than the body wall does. Accordingly, while the gage pressure of the gas in the lumen cannot overcome the flow resistance from the body wall, the gage pressure of the gas in the lumen can overcome the flow resistance from the body cavity.

It can be particularly beneficial to stop advancing the trocar at this point (e.g., as soon as the distal tip of the trocar has broken through the body wall) because farther advancement of the trocar into the body cavity may cause inadvertent damage to tissues and/or organs in the body cavity. Moreover, such damage may be quite difficult to remedy, and may require open surgery.

Again, it can be understood in view of the above description of the method 200 that it is beneficial to control the gage pressure of the gas in the lumen of the trocar resulting from step 240 within a narrow range of gage pressure. If the pressure of the gas in the lumen is too high, the gas may be undesirably injected into the body wall itself (e.g., between layers of the body wall, or within a layer of the body wall). Conversely, if the pressure of the gas in the lumen is too low, the user may not be able to visually observe a change when the distal tip of the trocar breaks through the body wall into the body cavity (as described further below).

In the context of the catheter placement device 100 described above, for example, the user can notice that the pressure of the gas in the lumen of the trocar has experienced a significant decrease by observing the position or movement of the shaft 134. In particular, movement of the shaft 134 farther into the housing 110 indicates that the compressed air from the bellows 130 and the lumen 182 of the catheter 180 has flowed out of the distal tip of the catheter 180. This movement of the shaft 134 can thereby be used to provide a visual notification to the user that the distal tip of the trocar has broken through the body wall and come into fluid communication with the body cavity.

The inventors performed multiple studies to identify a range of insufflation pressures at the midline and lateral points along the abdominal wall, as well as the initial insufflation pressure in cadaver models. The findings indicate that a Veress needle with pressures greater than 23.3 mmHg at the lateral sites and 20.7 mmHg at the midline locations (aside from M6 and L6) will result in preperitoneal insufflation. Conversely, a mean pressure of 15.51±4.15 mmHg is required to initially insufflate the peritoneal space.

Accordingly, the inventors have discovered that, in some embodiments, the optimal pressure of the gas in the bellows 130 and the lumen 182 of the catheter 180 (e.g., during steps 240 and 250 while the distal end of the lumen is sealed shut) is between 18.0 mmHg and 18.2 mmHg, or between 17.8 mmHg and 18.4 mmHg, or between 17.6 mmHg and 18.6 mmHg, or between 17.4 mmHg and 18.8 mmHg, or between 17.2 mmHg and 19.0 mmHg, or between 17.0 mmHg and 19.2 mmHg, or between 16.8 mmHg and 19.4 mmHg, or between 16.4 mmHg and 19.8 mmHg, or between 16 mmHg to 20 mmHg, without limitation. These ranges can be obtained, for example, by strategic selection of the spring rate of the spring 140 and the size of the internal space 131 of the bellows 130 (in combination with other factors such as the volume of the lumen 182, etc.).

At step 270, a surgical instrument is advanced through the lumen of the trocar and into the internal space of the body cavity. This step provides the end result of the method 200. That is, this step provides laparoscopic access to the body cavity space via the trocar. The method 200 attains this state in a controlled and safe manner, even if the user has only minimal medical training.

In the context of the catheter placement device 100 described above, to provide surgical instrument access to the body cavity space via the lumen 182 of the catheter 180, the valve member 124 can be removed from its engagement with the axle member 150. This can be performed by removing a retainer cap 152 (see FIGS. 4-6) from its engagement with the axle member 150, and then by pulling on the valve actuator knob 120 to remove the valve member 124 from within the axle member 150. With the valve member 124 removed from the axle member 150, one or more surgical instruments can be inserted into the lumen 182 via the axle member 150.

### OPTIONAL FEATURES AND ADDITIONAL EMBODIMENTS

While the rotary actuator 160 described above comprises a hand crank mechanism, in some embodiments the rotary actuator 160 can comprise a battery-operated motor, a spring mechanism, or other types of rotary actuators.

While the catheter 180 described above includes the helical protrusion 184, in some embodiments no such helical protrusion 184 is included. In some such embodiments, no rotary actuator is included. Instead, the catheter can be pushed into and just beyond the body wall.

In some embodiments, the rotary actuator 160 can become automatically locked when the gas pressure in the lumen experiences the decrease owing to the breaking through of the catheter through the body wall. For example, as shown in FIGS. 5 and 6, in some embodiments the bellow support member 132 includes one or more gear sectors 136. As the bellows 130 deflates and the bellow support member 132 correspondingly translates toward the axle member 150, the one or more gear sectors 136 can become forced into engagement with the driven bevel gear 186 and/or an idler bevel gear 112 that is meshed with the drive bevel gear 162. Since the one or more gear sectors 136 are fixed to the bellow support member 132, when the one or more gear sectors 136 mesh with the driven bevel gear 186 and/or the idler bevel gear 112 the driven bevel gear 186, the idler bevel gear 112, and the drive bevel gear 162 become restrained from additional rotary movement. In this manner, the rotary actuator 160 becomes automatically locked when the gas pressure in the lumen experiences the decrease resulting from the breaking through of the catheter through the body wall.

While the compressed air source described above comprises the bellows 130 and the spring 140, in some embodiments other types of compressed air sources can be used. For example, in some embodiments a small bottle of compressed carbon dioxide (CO2) can be used as the compressed air source. In some such embodiments, a pressure regulator can be included to control the gas pressure delivered from the bottle of compressed CO2. In some such embodiments, no such pressure regulator is required. Instead, the bottle of compressed CO2 is pressurized to the optimal pressure. In some embodiments, other types of compressed air sources can be used. Other types include, but are not limited to, a syringe, a compressible bulb, and the like. In some such embodiments, a pressure pop-off valve can be included to limit the maximum gas pressure generated from the syringe or the compressible bulb. Alternatively, in some embodiments a pressure gauge is provided that can be referenced by the user to establish a desired air pressure for the procedure.

In some embodiments, no valve member 124 is included. Instead, a mechanical latch can be included to retain the support member 132 in the position in which the bellows 130 is expanded and the spring 140 is compressed/loaded. When the time comes to pressurize the lumen 182 of the catheter 180 (as in step 240 of the method 200 described above), the mechanical latch can be released to allow the spring 140 to compress the bellows 130. Air from the bellows 130 will also flow into the lumen 182 of the catheter 180 to pressurize the air in the lumen 182.

In some embodiments, a proximal end portion of the catheter 180 can be separated from the rest of the catheter 180 to allow surgical instrument access via the lumen 182 of the catheter 180. For example, as indicated by the dotted line 185 in FIG. 5, in some embodiments the catheter 180 can be separated after the distal end portion of the catheter 180 is properly positioned relative to the body cavity (e.g., as an optional part of step 270 of the method 200 described above). The separation of the catheter 180 can be performed in various ways such as, but not limited to, the separation of a threaded connection at the dotted line 185, the removal of a collar at the dotted line 185, and/or by other mechanical disconnection means.

While this specification contains many specifics, these should not be construed as limitations on the scope of the disclosure or of what may be claimed, but rather as descriptions of features specific to particular implementations.

A number of embodiments have been described. Nevertheless, it will be understood that various modifications may be made without departing from the spirit and scope of the disclosure. Accordingly, other embodiments are within the scope of the following claims.

1. A medical device comprising:
a catheter defining a lumen extending along a longitudinal axis of the catheter;
a compressed gas source;
a valve member having: (i) a closed position in which the valve member blocks the compressed gas source from being in fluid communication with the lumen and (ii) an open position that allows fluid communication between the compressed gas source and the lumen; and
a rotary actuator that, when driven, rotates the catheter about its longitudinal axis.

2. The medical device of claim 1, wherein the catheter comprises a helical protrusion on an outer surface of the catheter.

3. The medical device of claim 1, wherein the rotary actuator comprises a hand crank by which the catheter is manually rotatable about its longitudinal axis.

4. The medical device of claim 1, wherein the valve member is manually movable between the closed and open positions.

5. The medical device of claim 1, wherein the compressed gas source comprises a bellows and a spring.

6. The medical device of claim 5, wherein the bellows and the spring are arranged such that the spring resists an expansion of the bellows to draw air into the bellows.

7. The medical device of claim 1, wherein the bellows is manually expandable.

8. The medical device of claim 1, wherein, when: (i) the bellows is expanded, (ii) the valve member is in the open position, and (iii) a distal end of the lumen is occluded, the spring compresses the bellows to create a gauge pressure of 16 mmHg to 20 mmHg within the lumen.

9. A method of accessing an internal region of a mammalian body, the method comprising:
making a skin incision;
placing a distal tip portion of a trocar through the skin incision and advancing the trocar a first distance into tissue;
after advancing the trocar the first distance, increasing a gas pressure in a lumen of the trocar;
after increasing the gas pressure in the lumen, advancing the trocar farther into the tissue until a decrease of the gas pressure in the lumen is noticeable, wherein the decrease of the gas pressure in the lumen indicates that the lumen has become in fluid communication with the internal region; and
advancing a surgical instrument through the lumen and into the internal region via the lumen of the trocar.

10. The method of claim 9, wherein the advancing the trocar into the tissue is performed by rotating the trocar about its longitudinal axis such that helical protrusions on an outer surface of the trocar drill into the tissue.

11. The method of claim 9, wherein the increasing the gas pressure in the lumen comprises opening a valve member to put a bellows and the lumen of the trocar in fluid communication with each other.

12. The method of claim 11, further comprising, prior to opening the valve member, expanding the bellows to: (i) cause gas to flow into the bellows and (ii) add loading to a spring.

13. The method of claim 12, further comprising, after expanding the bellows, closing the valve member prior to advancing the trocar farther into the tissue.

14. The method of claim 10, wherein the decrease of the gas pressure in the lumen causes the trocar to no longer be rotatable.

15. The method of claim 9, wherein the gas pressure is increased to a gauge pressure of 16 mmHg to 20 mmHg within the lumen of the trocar.

16. A trocar installation device comprising:
a trocar defining a lumen extending along a longitudinal axis of the trocar; and
a compressed gas source that is manually actuatable to selectively pressurize the lumen.

17. The trocar installation device of claim 16, wherein the trocar comprises a helical protrusion on an outer surface of the trocar, and wherein the trocar installation device further comprises a manual hand crank mechanism that is operable to rotate the trocar about its longitudinal axis.

18. The trocar installation device of claim 16, wherein the compressed gas source comprises a bellows and a spring.

19. The trocar installation device of claim 18, further comprising a latch mechanism that is operable to releasably detain the bellows in an expanded configuration and the spring in a loaded configuration.

20. The trocar installation device of claim 16, wherein the compressed gas source comprises a pressurized bottle containing the gas.
